(19) United States
(12) Patent Application Publication
(10) Pub. No.: US 2023/0223144 A1
(43) Pub. Date: Jul. 13, 2023

(54) RECONSTRUCTION OF SPARSE BIOMEDICAL DATA

(71) Applicant: X Development LLC, Mountain View, CA (US)

(72) Inventors: Garrett Raymond Honke, Mountain View, CA (US); Baihan Lin, New York, NY (US); Anupama Thubagere Jagadeesh, San Jose, CA (US)

(21) Appl. No.: 17/574,834

(22) Filed: Jan. 13, 2022

(51) Int. Cl. G16H 50/20; G16B 50/00; G16H 50/50; G16H 10/40

(52) U.S. Cl. CPC G16H 50/20; G16B 50/00; G16H 10/40; G16H 50/50

(57) ABSTRACT

The invention features a computer-implemented biological data prediction method executed by one or more processors including receiving, by the one or more processors, a biomedical data set comprising biomedical data corresponding to a plurality of detected analytes in a biological sample collected from a set of patients at intermittent time intervals, the biomedical data set having a first plurality of feature dimensions; processing, by the one or more processors, the biomedical data set to generate a low-rank tensor having a second plurality of feature dimensions, wherein the second plurality of feature dimensions can be lower than the first plurality of feature dimensions; generating, by the one or more processors, predicted biomedical data along the second plurality of feature dimensions corresponding to the intermittent time intervals; and creating a reconstructed biomedical data set including the predicted biomedical data and the biomedical data along the first plurality of feature dimensions.

### FIELD OF THE DISCLOSURE

The disclosure relates to factorizing sparse biomedical data into low-rank tensors to reconstruct the biomedical data.

### BACKGROUND

Healthcare entities, e.g., insurance company, doctor’s office, hospital, urgent care, or pharmacy, etc., store and manage biomedical data for many patients across a large number of sampling events and testing modalities. As an example, a patient interacting with one or more healthcare entities for the length of their life generates biomedical data across multiple data dimensions. An interaction with the healthcare entity can generate biomedical data including a height, weight, blood pressure, a respiration rate, analyte presence or quantities in a blood sample, etc. Medical records generated from these biomedical data are sparsely populated having unknown time intervals between sampling events resulting in a sparse time-dependent tensor of correlated data. The rate of biomedical data collection can be dependent on a patient’s health or age, e.g., a patient experiencing sickness or disease generates biomedical data at a higher rate. The sparsity, e.g., gaps, of the biomedical data prevents determining correlations and defines gaps in the medical records of individual patients as well as across large cohorts of patients.

### SUMMARY

In general, the disclosure relates to a predictive data system to factorize sparse biomedical data to infer missing values over a population of patients using robust principal component analysis (rPCA). Biomedical data, e.g., clinical data, are innately sparse, e.g., many missing values, since clinical tests and data collection are performed on biological samples collected at irregular time intervals using inconsistent methodologies. For example, the detection, diagnosis, and monitoring of a disease can include tracking concentrations of biomarkers and metabolites in collected fluid biological samples, such as patient fluid samples, across multiple collection modalities over extended time periods. This makes data aggregation and representation machine learning challenging, for example, in detecting time-dependent progression of disease.

The system solves this issue through tensor factorization in which the system receives a sparse data set, M, including biomedical and/or clinical data and de-convolves the data set into a representation featuring two tensors: a low-rank tensor, L, having lower rank than M and representing the decomposition of the data set into a concise number of latent representative dimensions; and a sparse tensor (or tensor), S, representing individual variations and outliers. The system uses an alternating minimum approach by optimizing L and S to minimize the reconstruction error of M. Determining an optimized L allows missing data in M to be imputed, e.g., predicted, along any feature dimension of the sparsely sampled original data. The low-rank tensor, L, includes interpretable insights about the relationships between different pairs of data dimensions. For example, in biomedical data collected from different subjects including biomarker volume information and associated timestamps, L includes three feature vectors corresponding to those three feature dimensions (e.g., subject, biomarker, timestamp), as well as how they interact.

In general, in a first aspect, the invention features a computer-implemented biological data prediction method executed by one or more processors including receiving, by the one or more processors, a biomedical data set comprising biomedical data corresponding to a plurality of detected analytes in a biological sample collected from a set of patients at intermittent time intervals, the biomedical data set having a first plurality of feature dimensions; processing, by the one or more processors, the biomedical data set to generate a low-rank tensor having a second plurality of feature dimensions, wherein the second plurality of feature dimensions can be lower than the first plurality of feature dimensions; generating, by the one or more processors, predicted biomedical data along the second plurality of feature dimensions corresponding to the intermittent time intervals; and creating a reconstructed biomedical data set including the predicted biomedical data and the biomedical data along the first plurality of feature dimensions.

Embodiments may include one or more of the following features. The generating can use principle component analysis. The principle component analysis can be robust principle component analysis. The processing can further include generating a sparse tensor having the second plurality of feature dimensions. The processing can further include calculating a reconstruction error of the low-rank tensor using an alternating minimum approach. Calculating the reconstruction error comprises can use the equation ||L||* + λ||S||1 such that M = L + S.

The method can include diagnosing a disease condition based on the predicted biomedical data set. The method can include communicating the disease condition for display. The plurality of detected analytes can be selected from the group consisting of a red blood cells, a white blood cells, a platelets, a sodium, a potassium, a magnesium, a nitrogen, a carbon dioxide, an oxygen, a glucose, a vitamin a, a vitamin d, a vitamin b 1 (thiamine), a vitamin b12, a folate, a calcium, a vitamin e, a vitamin k, a zinc, a copper, a vitamin b6, a vitamin c, a homocysteine, an iron, a hemoglobin, a hematocrit, an insulin, a melanin, a hormone, a testosterone, an estrogen, a cortisol, a thyroxine, a triiodothyronine, a human growth hormone, an insulin-like growth factor, a thyroid stimulating hormone (TSH), a carotenoid, a cytokine, an interleukin, a chloride, a cholesterol, a lipoprotein, a triglyceride, a c-peptide, a creatinine, a creatine, a creatine kinase, a urea, a ketone, a peptide, a protein, an albumin, a bilirubin, a myoglobin, an ESR, a CRP, an il6, an immunoglobin, a resistin, a ferritin, a transferrin, an antigen, a troponin, a gamma-glutamyltransferase (GGT), a lactate dehydrogenase (LD), an alanine aminotransferase, an alkaline phosphatase, or an aspartate aminotransferase. The method can include communicating the reconstructed biomedical data set for display.

In general, in a second aspect, the invention features a system including at least one processor; and a data store coupled to the at least one processor having instructions stored thereon which, when executed by the at least one processor, causes the at least one processor to perform operations including receiving, by the one or more processors, a biomedical data set comprising biomedical data corresponding to a plurality of detected analytes in a biological sample collected from a set of patients at intermittent time intervals, the biomedical data set having a first plurality of feature dimensions; processing, by the one or more processors, the biomedical data set to generate a low-rank tensor having a second plurality of feature dimensions, wherein the second plurality of feature dimensions can be lower than the first plurality of feature dimensions; generating, by the one or more processors, predicted biomedical data along the second plurality of feature dimensions corresponding to the intermittent time intervals; and creating a reconstructed biomedical data set including the predicted biomedical data and the biomedical data along the first plurality of feature dimensions.

Embodiments may include one or more of the following features. The generating can use principle component analysis. The principle component analysis can be robust principle component analysis. The operations can further include diagnosing a disease condition based on the predicted biomedical data set. The operations can further include providing, for display, a graphical user interface comprising: the disease condition based on the predicted biomedical data set. The operations can further include providing, for display, a graphical user interface comprising: a graphical representation of the reconstructed biomedical data set including the predicted biomedical data and the biomedical data along the first plurality of feature dimensions. The plurality of detected analytes can be selected from the group consisting of a red blood cells, a white blood cells, a platelets, a sodium, a potassium, a magnesium, a nitrogen, a carbon dioxide, an oxygen, a glucose, a vitamin a, a vitamin d, a vitamin b1 (thiamine), a vitamin b 12, a folate, a calcium, a vitamin e, a vitamin k, a zinc, a copper, a vitamin b6, a vitamin c, a homocysteine, an iron, a hemoglobin, a hematocrit, an insulin, a melanin, a hormone, a testosterone, an estrogen, a cortisol, a thyroxine, a triiodothyronine, a human growth hormone, an insulin-like growth factor, a thyroid stimulating hormone (TSH), a carotenoid, a cytokine, an interleukin, a chloride, a cholesterol, a lipoprotein, a triglyceride, a c-peptide, a creatinine, a creatine, a creatine kinase, a urea, a ketone, a peptide, a protein, an albumin, a bilirubin, a myoglobin, an ESR, a CRP, an IL6, an immunoglobin, a resistin, a ferritin, a transferrin, an antigen, a troponin, a gamma-glutamyltransferase (GGT), a lactate dehydrogenase (LD), an alanine aminotransferase, an alkaline phosphatase, or an aspartate aminotransferase. The processing can further include calculating a reconstruction error of the low-rank tensor using an alternating minimum approach. Calculating the reconstruction error can include using the equation ||L||* + λ||S||1 such that M = L + S.

Among other advantages, the predicted data imputed along sparse data dimensions reconstructs the medical history of a patient, or a cohort of patients, through time enabling discovery of heretofore unknown correlation patterns in the measured analytes that could serve as early warning indicators for disease.. Reconstructing the medical history of a single patient increases opportunities to create positive health outcomes and diagnosis of underlying diseases for a patient that may have been missed without the imputed data.

Additionally, reconstruction of patient data facilitates more effective analysis of by a patient, or healthcare provider. Treatment strategies can be tailored to the newly detected disease states or additional tests ordered based on trends introduced by the reconstruction.

Factorizing patient data into a low-rank representation includes determining a sparse tensor including data outliers from the original patient data. Removing biomedical data outliers from the original patient data tensor de-noises the original biomedical data providing a more accurate reconstruction of the historical patient data.

Other advantages will be apparent from the description, the drawings, and the claims.

### BRIEF DESCRIPTION OF THE DRAWINGS

FIG. 1 is a schematic representation of a system for reconstructing a patient data tensor with predicted biomedical data.

FIG. 2 is a schematic representation of the construction of a patient data tensor from received biomedical data.

FIG. 3 is a schematic representation of factorizing a matrix into a low - rank matrix and a sparse matrix.

FIG. 4 is a schematic representation of factorizing a low - rank matrix into a reconstructed matrix.

FIG. 5 is a work flow diagram of a process for reconstructing missing data from sparse patient data.

FIG. 6 is a schematic representation of a computing device.

In the figures, like symbols indicate like elements.

### DETAILED DESCRIPTION

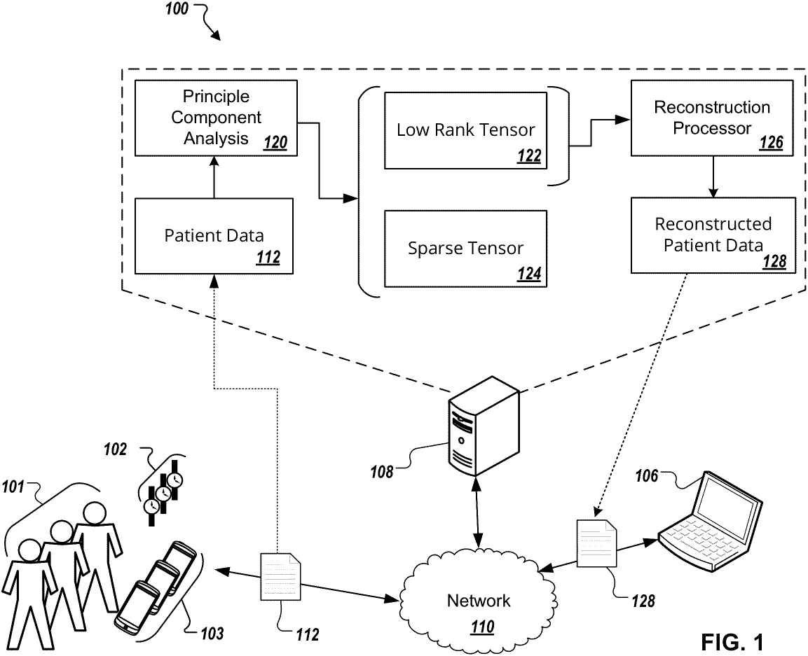

FIG. 1 is a diagram that illustrates an example system 100 for predicting mixing values of patient data over a population of patients using robust principal component analysis (rPCA). The system 100 includes a predictive data system (PDS) 108 in communication with a plurality of healthcare computing devices, such as wearable devices 102, user computing devices 103, or computing device 106 over a network 110. The network 110 can include public and/or private networks and can include the Internet.

The PDS 108 can include a system of one or more computers. In general, the PDS 108 is configured to perform one or more types of machine learning processes on a combination of time-dependent data, e.g., time-dependent psychological and/or biomedical data (collectively patient data 112) to impute missing data along any feature dimension of a complete representation of a patient’s data based on the data collected longitudinally, e.g., over time, over multiple collection events.

The PDS 108 obtains patient data over a period of time (e.g., a period of days, weeks, or months) including over multiple collection events. The patient data can include measurements of various biomedical parameters received from the healthcare computing devices. Wearable devices 102 monitor patient data such as, but not limited to, sleep onset latency, sleep duration, wake after sleep onset (WASO), heart rate, heart rate variability, blood pressure, blood pressure variability, daily step count, or any combination thereof.

Computing devices 103 are example user computing device, such as a cell phone, personal digital assistant, or tablet. A patient can provide patient data through the healthcare computing device 103 at self-directed or prescribed intervals.

In some implementations, data access rules executed by the PDS 108 permit the PDS 108 to obtain patient data 112 without third-party human interaction with the data on the PDS 108, thereby, protecting patient privacy. The PDS 108 can further protect each patient’s privacy by the PDS 108 assigning anonymized patient identifiers to each set of set of patients 101 whose data is obtained. The PDS 108 can use the anonymized patient identifiers to correlate data to specific patients while protecting personal information. For example, the system can remove personally identifiable information and assign a unique patient identifier to each unique patient. In some examples, the patient identifiers may be non-reversible to protect each patient’s identity. In some examples, the system can perform a cryptographic hash function on particular aspects of each patient’s identity, e.g., the system can hash a combination of the patient’s name, address, and date of birth to obtain a unique patient identifier.

Wearable devices 102 can be wearable computing devices, e.g., smart watches, health tracking devices, smart rings. Computing devices 103, 106 can be computing devices, e.g., mobile phones, smart phones, tablet computers, laptop computers, desktop computers, home assistant devices, or other portable or stationary computing device. Computing device 106 can be a computing device associated with a clinician (e.g., a psychologist or a psychiatrist) to which the PDS 108 transmits patient representations.

In various implementations, PDS 108 can perform some or all of the operations related to predicting missing biomedical data from patient data 112. For example, PDS 108 can include a PCA module 120, and a reconstruction processor 126. The PCA module 120 and reconstruction processor 126 can each be provided as one or more computer executable software modules or hardware modules. That is, some or all of the functions of PCA module 120 and reconstruction processor 126 can be provided as a block of code, which upon execution by a processor, causes the processor to perform functions described below. Some or all of the functions of PCA module 120 and reconstruction processor 126 can be implemented in electronic circuitry, e.g., as field programmable gate array (FPGA) or an application specific integrated circuit (ASIC).

In operation, PDS 108 collects patient data 112 from a set of set of patients 101. The patient data 112 is provided to PDS 108 over the network 110. More specifically, patient data 112 can include multiple time dependent streams or “channels” of different types of patient biomedical data.

The PDS 108 then applies a series of one or more machine learning algorithms to the patient data 112 to generate a low-rank representation of the biomedical data. The PDS 108 includes a principle component analysis (PCA) module 120 which stores and executes one or more machine learning algorithms, such as principle component algorithms. The PCA module 120 processes the patient data 112 to generate a biomedical data representation.

For example, the PDS 108 can receive patient biomedical data from the set of patients 101 wearable devices 102 over intermittent time intervals (e.g., days, weeks, months). Biomedical data can include, but is not limited to, measurements of patient biomedical characteristics such as sleep onset latency, sleep duration, wake after sleep onset (WASO), heart rate, heart rate variability, daily step count, or any combination thereof. The PDS 108 can receive uploads of biomedical data from the wearable devices 102 of patient’s 101 who have opted-in to the PDS 108 analysis, e.g., at the advice or with the assistance of a clinician. The PDS 108 can receive regular (e.g., daily, weekly, monthly) uploads of patient biomedical data that includes periodic measurements of the various biomedical characteristics noted above.

On the whole, the PDS 108 can receive multiple channels of both patient biomedical data and patient EMA data each day for a period of weeks, months, or years. Moreover, the particular types of biomedical data and EMA data collected may differ for each particular patient dependent on the patient’s circumstances. A clinician may be permitted to select particular patient data types for processing by the PDS 108 for each of their patients

For example, the PDS 108 may accumulate patient data 112 over the course of several days or weeks before analyzing the patient data 112 using the PCA module 120 and the reconstruction processor 126, e.g., to ensure sufficient data is available to predicted biomedical data for the set of patients 101. Once sufficient patient data 112 has been collected for a particular set of set of patients 101, the PDS 108 can update the analysis of the set of patients 101 data at regular intervals (e.g., daily, weekly, or month) by incorporating the data received over the time interval with the set of patients 101 past biomedical data. The collected patient data 112 can be correlated with a patient, a patient identifier, or the collected patient data in the patient data tensor 200 can be anonymized by the PDS 108, e.g., patient data decorrelated with identifying information. Alternatively, the patient data 112 is anonymized before the PDS 108 receives the patient data 112.

The PDS 108 applies the patient data 112 as input to the PCA module 120. The PCA module 120 executes a principle component analysis algorithm to generate a low-rank tensor 122 and a sparse tensor 124. The low-rank tensor 122 forms a low-rank representation of the received patient data 112 and the sparse tensor 124 includes outliers from the patient data 112. The sparse tensor 124 is discarded by the PDS 108 which inputs the low-rank tensor 122 into a reconstruction processor 126. The reconstruction processor 126 which reconstructs a representation of the original patient data 112 as reconstructed patient data 128 from the low-rank tensor 122.

The reconstruction processor 126 generates predicted biomedical data using the low-rank tensor 122 and the sparse tensor 124 and reconstructs the patient data 112 into reconstructed patient data 128 which includes predicted biomedical data. The reconstruction processor 126 constructs the predicted biomedical data into the patient data 112 where there are empty elements along the dimensions of the patient data 112. The reconstructed patient data 128 includes predicted biomedical data along one or more feature dimensions.

The PDS 108 stores the reconstructed patient data 128 for access from one or more devices, e.g., healthcare computing devices 106, user computing devices 103, or wearable devices 102, over the network 110. Alternatively, the PDS 108 can send the reconstructed patient data 128 over the network 110 to the healthcare computing devices 106, wearable devices 102, or user computing devices 103 for access by and/or display to individual users or clinicians.

The reconstructed patient data 128 contains the patient data 112 and the predicted biomedical data. From the reconstructed patient data 128, information relating to interpretable insights into the health status of a user, or the collective health status of the set of patients 101, can be determined by further processing or access by a healthcare entity, such as an insurance company, doctor’s office, hospital, urgent care, or pharmacy. As an example, a doctor may determine that a patient of the set of patients 101 has a previously undiagnosed disease based on the reconstructed patient data 128. As a second example, a hospital may determine that one or more patients of the set of patients 101 requires additional testing, e.g., testing frequency or testing modes, based on interpreting the reconstructed patient data 128.

In some implementations, the PDS 108 includes a disease detection module which receives the reconstructed patient data 128 and determines a disease state from the data. In some examples, the disease detection module can confirm an existing disease diagnosis, or the disease detection module can determine a new disease diagnosis. The disease diagnosis can depend on the feature dimensions of the reconstructed patient data 128.

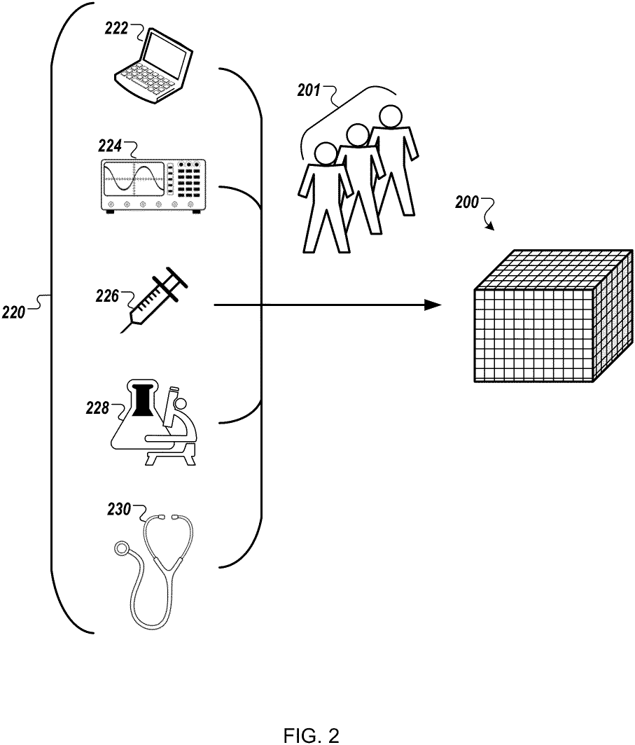

The patient data 112 is constructed from biomedical data collected from a set of patients 101. A user, or set of patients 101, generates the biomedical data when interacting with a healthcare entity. Referring to FIG. 2, a schematic illustration of the construction of a patient data tensor 200, e.g., patient data 112, from biomedical data 220 generated by a set of patients 201, e.g., set of patients 101, is shown. In some implementations, the patient data tensor 200 is constructed from biomedical data related to a single patient. In alternative implementations, the patient data tensor 200 is constructed from patient data related to more than one patient, such as the set of patients 201.

A tensor is a data construct including a number of dimensions along which data is populated. A tensor rank, e.g., order, or degree, is a parameter of the tensor which describes the number of dimensions of the underlying space. As an example, a vector is a rank 1 tensor, e.g., a series of values along a single dimension. A single medical test performed on a single patient over time would generate a vector of data, e.g., the result of the tests concatenated through time. Further, a two-dimensional matrix is a rank 2 tensor. Increasing tensor rank corresponds to increasing tensor dimensionality.

In the example of FIG. 2, the dimensionality of patient data tensor 200 corresponds to the number of feature dimensions of the collected biomedical data from the set of patients 201, as described above. As such, the patient data tensor 201 has a rank corresponding to the maximal number of linearly independent columns of feature dimensions of the patient data tensor 201. In implementations in which there are more features dimensions than subjects or trial instances, then the rank is smaller than the number of feature dimensions.

As a set of patients 210 interacts with a healthcare entity, biomedical data 220 is generated. A healthcare entity collects the biomedical data 220 independently over a time period. For example, the biomedical data 220 can include a medical professional report 222, a health monitoring report 224, a test result 226, a lab result 228, or a health check result 230. The time period over which the biomedical data 220 is collected can be different for each example, or it can be the same.

The biomedical data 220 collected from the reports 222 or 224, or results 226, 228, or 230 can include one or more common feature dimensions, such as time, and/or one or more independent feature dimensions. For example, the test result 226 may not be correlated with the health monitoring report 224, or the medical professional report 222 may not be associated with the lab result 228. The biomedical data 220 is represented by a patient data tensor 200 examples of which include a vector, a matrix, or a tensor. For example, a blood pressure collected from a single patient over time can be represented as a vector. In a second example, a large number of biomarkers detected in a single test result 226 from a single patient over time can be represented as a matrix. Biomedical data 220 from multiple patients, such as the set of patients 201, can be constructed into additional dimensions of the patient data tensor 200.

Examples of feature dimensions of biomedical data 220 which can be included in the patient data tensor 200 can include the following: the medical professional report 222 may include feature dimensions such as height, weight, blood pressure, respiration rate, heart rate, or gender, while the test result 226 or lab result 228 may include dimensions such as biomarker presence or quantity, cholesterol level, gene presence or activity, or metabolite presence or quantity.

In some implementations, the biomedical data 220 includes the presence, quantity, or volume of an analyte present in a biological sample collected from a patient at a time point or series of time points. Examples of analytes can include red blood cells, white blood cells, platelets, sodium, potassium, magnesium, nitrogen, carbon dioxide, oxygen, glucose, Vitamin A, Vitamin D, Vitamin B1 (thiamine), Vitamin B12, folate, calcium, Vitamin E, Vitamin K, zinc, copper, Vitamin B6, Vitamin C, homocysteine, iron, hemoglobin, hematocrit, insulin, melanin, hormones, testosterone, estrogen, cortisol, thyroxine, triiodothyronine, human growth hormone, insulin-like growth factors, thyroid stimulating hormone (TSH), carotenoids, cytokines, interleukins, chlorides, cholesterols, lipoproteins, triglycerides, c-peptide, creatinine, creatine, creatine kinase, urea, ketones, peptides, proteins, albumin, bilirubin, myoglobin, ESR, CRP, IL6, immunoglobins, resistin, ferritin, transferrin, antigens, troponins, gamma-glutamyltransferase (GGT), lactate dehydrogenase (LD), alanine aminotransferase, alkaline phosphatase, and aspartate aminotransferase.

The biomedical data 220 collected at irregular intervals results in sparse data population of the patient data tensor 200. The biomedical data 220 representations, such as a vector of blood pressure data or tensor of biomarker data, concatenated together across a common timeline generates the patient data tensor 200. A sparse data structure, such as patient data tensor 200, contains discrete data along one or more feature dimensions of the tensor, and is empty (e.g., no data) in between the discrete data points along the one or more dimensions.

The computer program stored in the PDS 108, or on an alternative computer attached to the network 110, constructs the biomedical data 220 into the patient data tensor 200 preserving the data along all of the collected dimensions. For example, biomedical data 220 including patient records timestamped at certain time points transformed into a tabular structure including rows corresponding to the patient record timestamps, and columns corresponding to indices of the analytes.

Each element includes the value of the analyte level at the corresponding timestamp and analyte index. Reconstructing the biomedical data 220 into a matrix is performed by separating the records by timestamp, and generating T matrices corresponding to T time points. The corresponding matrices share feature dimension number M and patient dimension number N. Concatenating the matrices into a data structure creates a data tensor of M x N x T. The patient data tensor 200 is used as patient data 112 in the system of FIG. 1.

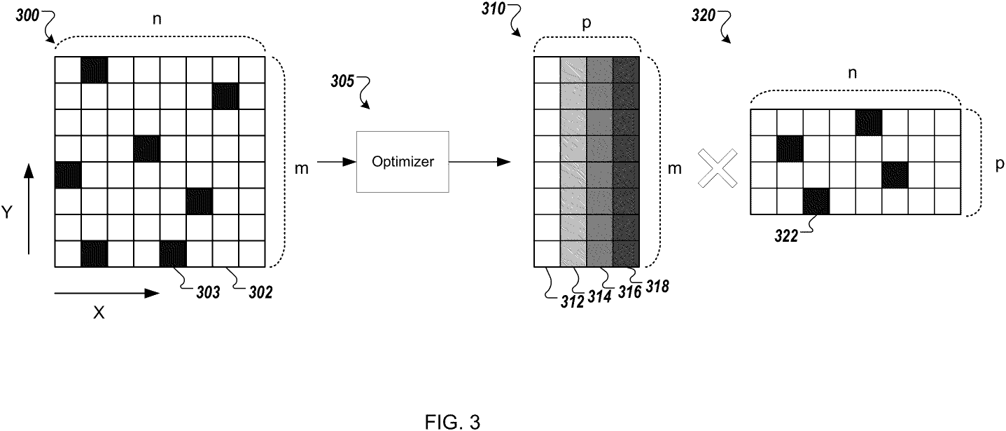

The patient data 112 is processed by the PCA module 120 into a low-rank tensor 122 and the sparse tensor 124. FIG. 3 is a schematic illustration of reducing a matrix 300, which is an example of a tensor of rank 2 and representative of the patient data 112, to a low-rank matrix 310, having lower rank than matrix 300, and a sparse matrix 320, such as low-rank tensor 122 and sparse tensor 124, respectively. The example of FIG. 3 includes a two-dimensional matrix 300, but in general the matrix 300 can include any number of dimensions.

The matrix 300 is a two-dimensional matrix having a number of elements 302 along a first dimension and a second dimension, e.g., X and Y. As shown, matrix 300 includes m elements along the Y dimension and n elements along the X dimension. A number of elements 302 in the matrix 300 include values 303 (e.g., black elements 303) while a second set of elements 302 include no values (e.g., white elements 302).

In one example, each column of the n columns along the X dimension of matrix 300 corresponds to a distinct timestamp, such as a series of dates on which biomedical data was taken. Each row of the m rows of the Y dimension of matrix 300 is a unique patient, such as a single patient of the set of set of patients 101. The elements 302 at each row and column position can include values 303 corresponding to a received biomedical data from the corresponding patient at a timestamp.

The collection of biomedical data is intermittent and varies between patients, setting, and collection method. For example, a first patient may provide biomedical data more frequently than a second, while a second patient may only provide biomedical data once along the range of the X dimension. This results in a sparse matrix 300 having few values 303 in corresponding elements 302.

In general, the optimizer engine 305 can use an optimization algorithm capable of reducing the reconstruction error of the matrix 300. For example, general optimization algorithms can include gradient descent, or evolutionary algorithms. In some implementations, an optimizer engine 305 uses an alternating minimization approach to optimize both the low-rank matrix (L) 310 and sparse matrix (S) 320. The optimizer engine 305 optimizes the low-rank matrix (L) 310 by minimizing the rank of L. The optimizer engine 305 determines a low-rank matrix 310 and a sparse matrix 320 and reconstructs an intermediate tensor M*. The optimizer engine 305 determines a reconstruction error value based on the intermediate tensor M* and the matrix 300.

The sparse reconstruction error is calculated with the equation ∥L∥* + λ∥S∥₁ such that M = L + S. The matrix function ∥L∥* calculates the rank of the L; ||S||₁ is the entry-wise ℓ₁ norm, which sums the magnitudes of the component vectors of S; and X > 0 is a regularization parameter which the PCA module 120 balances during calculation of L and S. The PCA module 120 performs calculations to vary the values of low-rank matrix 310, the values of sparse matrix 320, and λ until a reconstruction error value surpasses a reconstruction error threshold.

The optimizer engine 305 processes the matrix 300 in an unsupervised manner to generate the low-rank matrix 310 and the sparse matrix 320. The sparse matrix 320 includes a number of outlier values 322 in a substantially empty matrix, while low-rank matrix 310 includes a number of vectors, e.g., vectors 312, 314, 316, and 318. The outlier values 322 are determined through the alternating minimization process. The outlier values 322 provide the lowest reconstruction error value of M*.

The reconstruction error value can be considered a partial reconstruction error given that the matrix 300 is incomplete, e.g., is partially complete having few values 303 in corresponding elements 302 and multiple empty elements 302. As such, the reconstruction error value of M* is calculated between the elements 302 of matrix 300 containing values and the corresponding elements of M*. The reconstruction error can be calculated using a general difference algorithm, such as a mean squared error algorithm.

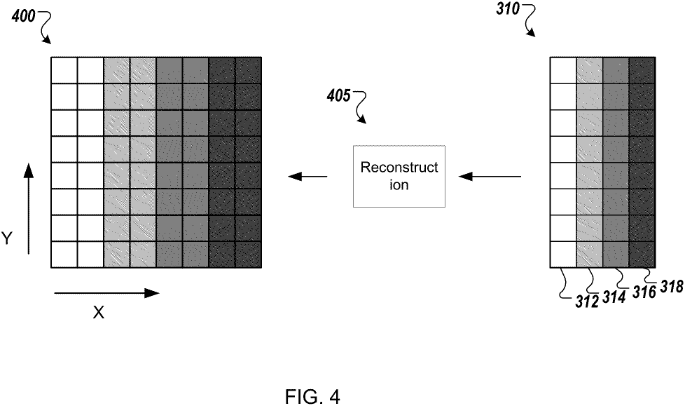

The vectors 312, 314, 316, and 318 are used as linear combination basis vectors in the reconstruction of the matrix 300, described later with reference to FIG. 4. The low-rank matrix 310 is a matrix of lower rank than the matrix 300, e.g., has fewer dimensions, e.g., p < n. The dimensions of the low-rank matrix 310 may be significantly lower than those of the matrix 300. For example, matrix 300 is an m × n matrix, low-rank matrix 310 is an m × p matrix, and sparse matrix 320 is a p × n matrix where p is less than both m and n.

The values 322 of the sparse matrix 320 are outliers having a magnitude above an error threshold with respect to the average values of the respective dimensions. When the optimizer engine 305 determines that the reconstruction error value surpasses a reconstruction error threshold, optimizer engine 305 outputs the low-rank matrix 310 and the sparse matrix 320.

To generate the reconstructed patient data 128 including imputed data between the data points of patient data 112, the reconstruction processor 126 receives the low-rank tensor 122 and constructs the reconstructed patient data 128. FIG. 4 is a schematic diagram of an example reconstruction process that the reconstruction processor 126 follows in reconstructing the low-rank tensor 122 and the sparse tensor 124 into the reconstructed patient data 128.

A reconstruction engine 405 receives the low-rank matrix 310 including basis vectors 312, 314, 316, and 318. The reconstruction engine 405 uses the low - rank matrix 310 to reconstruct a reconstructed matrix 400 which includes data imputed, e.g., predicted, from the low - rank matrix 310. The reconstructed matrix 400 containing the imputed data (shaded) has the same dimensions, e.g., X and Y, as the original matrix 300 before factorization. The reconstructed matrix 400 includes the same number of elements along each dimension as matrix 300, e.g., n elements along X, and m elements along Y.

The n x m reconstructed matrix 400 is a representation of the matrix 300 which includes continuous data along the dimensions X and Y. As an example, whereas the original matrix 300 included sparse data along X and Y, having many empty elements, reconstructed matrix 400 includes data along all elements of X and Y. In the example of FIG. 1, reconstructed patient data 128 includes continuously reconstructed data along all the dimensions of the reconstructed patient data 128 tensor.

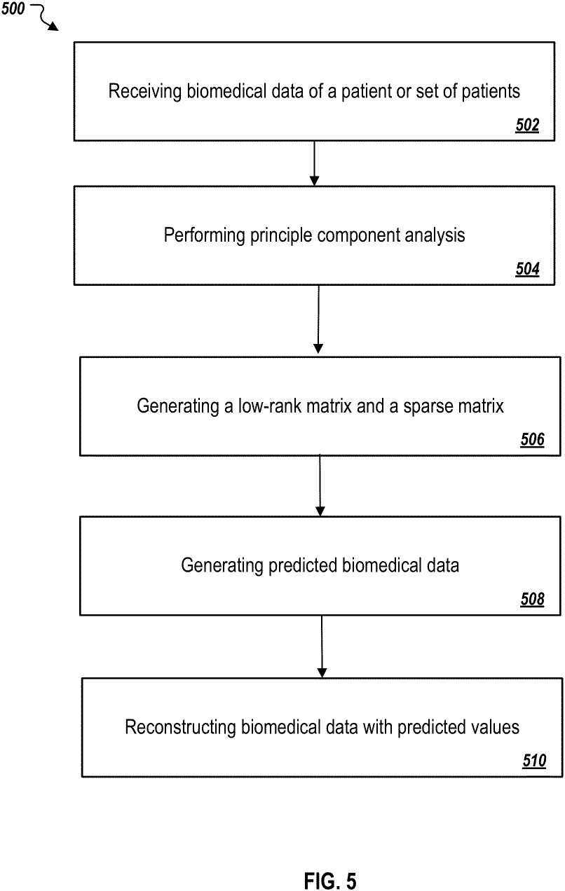

FIG. 5 is a flow-chart diagram of the individual steps for predicting biomedical data values in sparse biomedical data records (500). A PDS 108 receives patient data 112 collected from a set of patients 101 (502). The patient data 112 can be collected from one or more patients included in the set of patients 101 across a number of dimensions relating to the collected biomedical data. The patient data 112 can be received from any appropriate source of biomedical data such as a healthcare entity, a laboratory, a self-reporting patient, or a data aggregating business.

The PDS 108 inputs the received patient data 112 into a PCA module 120 which performs calculations on the patient data 112 using one or more stored algorithms. In some implementations, the PCA module 120 stores an rPCA algorithm which the PCA module 120 performs on the patient data 112 (504). The PCA module 120 can perform alternative tensor decomposition algorithms on the patient data 112, such as tensor rank decomposition, higher-order singular value decomposition, Tucker decomposition, matrix product states, or block term decomposition..

The PCA module 120 generates a low-rank tensor 122 and a sparse tensor 124 based on the patient data 112 (506). The low-rank tensor 122 is a tensor of lower rank than the patient data 112 and includes a number of basis tensors from which the patient data 112 can be reconstructed. The sparse tensor 124 is a tensor containing data points which are determined to be outliers from the patient data 112. In some implementations, the sparse tensor 124 is discarded before the PCA module 120 transmits the low-rank tensor 122 for reconstruction. In alternative implementations, the PCA module 120 stores or transmits the sparse tensor 124 for further processing, such as during reconstruction.

The PCA module 120 transmits the low-rank tensor 122 to a reconstruction processor 126. The reconstruction processor 126 receives the low-rank tensor 122 and generates predicted biomedical data along one or more dimensions of the patient data 112 (508). The predicted data corresponds to time intervals between the intermittently collected biomedical data present in the patient data 112.

The reconstruction processor 126 reconstructs a representation of the patient data 112 as reconstructed patient data 128 (510) using the predicted biomedical data. In some implementations, the reconstruction processor 126 receives the sparse tensor 124 to perform the reconstruction of the reconstructed patient data 128.

The PCA module 120 receives the reconstructed patient data 128 from the reconstruction processor 126. Optionally, the PCA module 120 can transmit the reconstructed patient data 128 to one or more networked computing devices, such as healthcare computing devices 106, for display to a user or analysis by a healthcare entity.

In general, the patients referred to throughout the description are human patients, though this is not necessary. In some implementations, the patients are animal patients and the healthcare entity can further include veterinary-related entities, services, and locations.

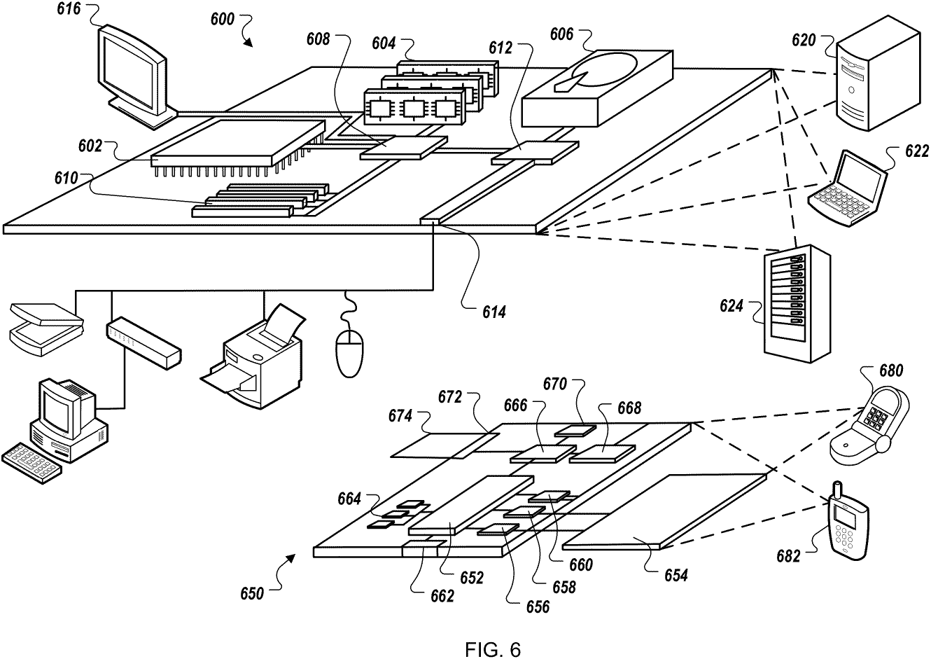

As noted previously, the systems and methods disclosed above utilize data processing apparatus to implement aspects of the process to factorize and reconstruct patient data described herein. FIG. 6 shows an example of a computing device 600 and a mobile computing device 650 that can be used as data processing apparatuses to implement the techniques described here. The computing device 600 is intended to represent various forms of digital computers, such as laptops, desktops, workstations, personal digital assistants, servers, blade servers, mainframes, and other appropriate computers. The mobile computing device 650 is intended to represent various forms of mobile devices, such as personal digital assistants, cellular telephones, smart-phones, and other similar computing devices. The components shown here, their connections and relationships, and their functions, are meant to be examples only, and are not meant to be limiting.

The computing device 600 includes a processor 602, a memory 604, a storage device 606, a high-speed interface 608 connecting to the memory 604 and multiple high-speed expansion ports 610, and a low-speed interface 612 connecting to a low-speed expansion port 614 and the storage device 606. Each of the processor 602, the memory 604, the storage device 606, the high-speed interface 608, the high-speed expansion ports 610, and the low-speed interface 612, are interconnected using various busses, and may be mounted on a common motherboard or in other manners as appropriate. The processor 602 can process instructions for execution within the computing device 600, including instructions stored in the memory 604 or on the storage device 606 to display graphical information for a GUI on an external input/output device, such as a display 616 coupled to the high-speed interface 608. In other implementations, multiple processors and/or multiple buses may be used, as appropriate, along with multiple memories and types of memory. Also, multiple computing devices may be connected, with each device providing portions of the necessary operations (e.g., as a server bank, a group of blade servers, or a multi-processor system).

The memory 604 stores information within the computing device 600. In some implementations, the memory 604 is a volatile memory unit or units. In some implementations, the memory 604 is a non-volatile memory unit or units. The memory 604 may also be another form of computer-readable medium, such as a magnetic or optical disk.

The storage device 606 is capable of providing mass storage for the computing device 600. In some implementations, the storage device 606 may be or contain a computer-readable medium, such as a floppy disk device, a hard disk device, an optical disk device, or a tape device, a flash memory or other similar solid state memory device, or an array of devices, including devices in a storage area network or other configurations. Instructions can be stored in an information carrier. The instructions, when executed by one or more processing devices (for example, processor 602), perform one or more methods, such as those described above. The instructions can also be stored by one or more storage devices such as computer- or machine-readable mediums (for example, the memory 604, the storage device 606, or memory on the processor 602).

The high-speed interface 608 manages bandwidth-intensive operations for the computing device 600, while the low-speed interface 612 manages lower bandwidth-intensive operations. Such allocation of functions is an example only. In some implementations, the high-speed interface 608 is coupled to the memory 604, the display 616 (e.g., through a graphics processor or accelerator), and to the high-speed expansion ports 610, which may accept various expansion cards (not shown). In the implementation, the low-speed interface 612 is coupled to the storage device 606 and the low-speed expansion port 614. The low-speed expansion port 614, which may include various communication ports (e.g., USB, Bluetooth, Ethernet, wireless Ethernet) may be coupled to one or more input/output devices, such as a keyboard, a pointing device, a scanner, or a networking device such as a switch or router, e.g., through a network adapter.

The computing device 600 may be implemented in a number of different forms, as shown in the figure. For example, it may be implemented as a standard server 620, or multiple times in a group of such servers. In addition, it may be implemented in a personal computer such as a laptop computer 622. It may also be implemented as part of a rack server system 624. Alternatively, components from the computing device 600 may be combined with other components in a mobile device (not shown), such as a mobile computing device 650. Each of such devices may contain one or more of the computing device 600 and the mobile computing device 650, and an entire system may be made up of multiple computing devices communicating with each other.

The mobile computing device 650 includes a processor 652, a memory 664, an input/output device such as a display 654, a communication interface 666, and a transceiver 668, among other components. The mobile computing device 650 may also be provided with a storage device, such as a micro-drive or other device, to provide additional storage. Each of the processor 652, the memory 664, the display 654, the communication interface 666, and the transceiver 668, are interconnected using various buses, and several of the components may be mounted on a common motherboard or in other manners as appropriate.

The processor 652 can execute instructions within the mobile computing device 650, including instructions stored in the memory 664. The processor 652 may be implemented as a chipset of chips that include separate and multiple analog and digital processors. The processor 652 may provide, for example, for coordination of the other components of the mobile computing device 650, such as control of user interfaces, applications run by the mobile computing device 650, and wireless communication by the mobile computing device 650.

The processor 652 may communicate with a user through a control interface 658 and a display interface 656 coupled to the display 654. The display 654 may be, for example, a TFT (Thin-Film-Transistor Liquid Crystal Display) display or an OLED (Organic Light Emitting Diode) display, or other appropriate display technology. The display interface 656 may comprise appropriate circuitry for driving the display 654 to present graphical and other information to a user. The control interface 658 may receive commands from a user and convert them for submission to the processor 652. In addition, an external interface 662 may provide communication with the processor 652, so as to enable near area communication of the mobile computing device 650 with other devices. The external interface 662 may provide, for example, for wired communication in some implementations, or for wireless communication in other implementations, and multiple interfaces may also be used.

The memory 664 stores information within the mobile computing device 650. The memory 664 can be implemented as one or more of a computer-readable medium or media, a volatile memory unit or units, or a non-volatile memory unit or units. An expansion memory 674 may also be provided and connected to the mobile computing device 650 through an expansion interface 672, which may include, for example, a SIMM (Single In Line Memory Module) card interface. The expansion memory 674 may provide extra storage space for the mobile computing device 650, or may also store applications or other information for the mobile computing device 650. Specifically, the expansion memory 674 may include instructions to carry out or supplement the processes described above, and may include secure information also. Thus, for example, the expansion memory 674 may be provide as a security module for the mobile computing device 650, and may be programmed with instructions that permit secure use of the mobile computing device 650. In addition, secure applications may be provided via the SIMM cards, along with additional information, such as placing identifying information on the SIMM card in a non-hackable manner.

The memory may include, for example, flash memory and/or NVRAM memory (non-volatile random access memory), as discussed below. In some implementations, instructions are stored in an information carrier. The instructions, when executed by one or more processing devices (for example, processor 652), perform one or more methods, such as those described above. The instructions can also be stored by one or more storage devices, such as one or more computer- or machine-readable mediums (for example, the memory 664, the expansion memory 674, or memory on the processor 652). In some implementations, the instructions can be received in a propagated signal, for example, over the transceiver 768 or the external interface 662.

The mobile computing device 650 may communicate wirelessly through the communication interface 666, which may include digital signal processing circuitry where necessary. The communication interface 666 may provide for communications under various modes or protocols, such as GSM voice calls (Global System for Mobile communications), SMS (Short Message Service), EMS (Enhanced Messaging Service), or MMS messaging (Multimedia Messaging Service), CDMA (code division multiple access), TDMA (time division multiple access), PDC (Personal Digital Cellular), WCDMA (Wideband Code Division Multiple Access), CDMA2000, or GPRS (General Packet Radio Service), among others. Such communication may occur, for example, through the transceiver 668 using a radio-frequency. In addition, short-range communication may occur, such as using a Bluetooth, Wi-Fi, or other such transceiver (not shown). In addition, a GPS (Global Positioning System) receiver module 670 may provide additional navigation- and location-related wireless data to the mobile computing device 650, which may be used as appropriate by applications running on the mobile computing device 650.

The mobile computing device 650 may also communicate audibly using an audio codec 660, which may receive spoken information from a user and convert it to usable digital information. The audio codec 660 may likewise generate audible sound for a user, such as through a speaker, e.g., in a handset of the mobile computing device 650. Such sound may include sound from voice telephone calls, may include recorded sound (e.g., voice messages, music files, etc.) and may also include sound generated by applications operating on the mobile computing device 650.

The mobile computing device 650 may be implemented in a number of different forms, as shown in the figure. For example, it may be implemented as a cellular telephone 680. It may also be implemented as part of a smart-phone 682, personal digital assistant, or other similar mobile device.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms machine-readable medium and computer-readable medium refer to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term machine-readable signal refers to any signal used to provide machine instructions and/or data to a programmable processor.

To provide for interaction with a user, the systems and techniques described here can be implemented on a computer having a display device (e.g., an OLED (organic light emitting diode) display or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (LAN), a wide area network (WAN), and the Internet.

The computing system can include clients and servers. A client and server are generally remote from each other and typically interact through a communication network. The relationship of client and server arises by virtue of computer programs running on the respective computers and having a client-server relationship to each other.

In some embodiments, the computing system can be cloud based and/or centrally processing data. In such case anonymous input and output data can be stored for further analysis. In a cloud based and/or processing center set-up, compared to distributed processing, it can be easier to ensure data quality, and accomplish maintenance and updates to the calculation engine, compliance to data privacy regulations and/or troubleshooting.

A number of implementations have been described. Other implementations are in the following claims.

What is claimed is:

1. A computer-implemented biological data prediction method executed by one or more processors and comprising:
receiving, by the one or more processors, a biomedical data set comprising biomedical data corresponding to a plurality of detected analytes in a biological sample collected from a set of patients at intermittent time intervals, the biomedical data set having a first plurality of feature dimensions;
processing, by the one or more processors, the biomedical data set to generate a low-rank tensor having a second plurality of feature dimensions, wherein the second plurality of feature dimensions is lower than the first plurality of feature dimensions; and
generating, by the one or more processors, predicted biomedical data along the second plurality of feature dimensions corresponding to the intermittent time intervals; and
creating a reconstructed biomedical data set including the predicted biomedical data and the biomedical data along the first plurality of feature dimensions.

2. The method of claim 1, wherein the generating uses principle component analysis.

3. The method of claim 2, wherein the principle component analysis is robust principle component analysis.

4. The method of claim 1, wherein the processing further comprises generating a sparse tensor having the second plurality of feature dimensions.

5. The method of claim 1, wherein the processing further comprises calculating a reconstruction error of the low-rank tensor using an alternating minimum approach.

6. The method of claim 5, wherein calculating the reconstruction error comprises using the equation ||L||* + λ||S||₁ such that M = L + S.

7. The method of claim 1, further comprising diagnosing a disease condition based on the predicted biomedical data set.

8. The method of claim 1, wherein the plurality of detected analytes are selected from the group consisting of a red blood cells, a white blood cells, a platelets, a sodium, a potassium, a magnesium, a nitrogen, a carbon dioxide, an oxygen, a glucose, a Vitamin A, a Vitamin D, a Vitamin B1 (thiamine), a Vitamin B12, a folate, a calcium, a Vitamin E, a Vitamin K, a zinc, a copper, a Vitamin B6, a Vitamin C, a homocysteine, an iron, a hemoglobin, a hematocrit, an insulin, a melanin, a hormone, a testosterone, an estrogen, a cortisol, a thyroxine, a triiodothyronine, a human growth hormone, an insulin-like growth factor, a thyroid stimulating hormone (TSH), a carotenoid, a cytokine, an interleukin, a chloride, a cholesterol, a lipoprotein, a triglyceride, a c-peptide, a creatinine, a creatine, a creatine kinase, a urea, a ketone, a peptide, a protein, an albumin, a bilirubin, a myoglobin, an ESR, a CRP, an IL6, an immunoglobin, a resistin, a ferritin, a transferrin, an antigen, a troponin, a gamma-glutamyltransferase (GGT), a lactate dehydrogenase (LD), an alanine aminotransferase, an alkaline phosphatase, or an aspartate aminotransferase.

9. The method of claim 1, further comprising communicating the reconstructed biomedical data set for display.

10. The method of claim 7, further comprising communicating the disease condition for display.

11. A system comprising:
at least one processor; and a data store coupled to the at least one processor having instructions stored thereon which, when executed by the at least one processor, causes the at least one processor to perform operations comprising:
receiving, by the one or more processors, a biomedical data set comprising biomedical data corresponding to a plurality of detected analytes in a biological sample collected from a set of patients at intermittent time intervals, the biomedical data set having a first plurality of feature dimensions;
processing, by the one or more processors, the biomedical data set to generate a low-rank tensor having a second plurality of feature dimensions, wherein the second plurality of feature dimensions is lower than the first plurality of feature dimensions; and
generating, by the one or more processors, predicted biomedical data along the second plurality of feature dimensions corresponding to the intermittent time intervals; and
creating a reconstructed biomedical data set including the predicted biomedical data and the biomedical data along the first plurality of feature dimensions.

12. The system of claim 11, wherein the generating uses principle component analysis.

13. The system of claim 12, wherein the principle component analysis is robust principle component analysis.

14. The system of claim 11, wherein the operations further comprise diagnosing a disease condition based on the predicted biomedical data set.

15. The system of claim 14, wherein the operations further comprise providing, for display, a graphical user interface comprising:
the disease condition based on the predicted biomedical data set.

16. The system of claim 11, wherein the operations further comprise providing, for display, a graphical user interface comprising:
a graphical representation of the reconstructed biomedical data set including the predicted biomedical data and the biomedical data along the first plurality of feature dimensions.

17. The system of claim 11, wherein the plurality of detected analytes are selected from the group consisting of a red blood cells, a white blood cells, a platelets, a sodium, a potassium, a magnesium, a nitrogen, a carbon dioxide, an oxygen, a glucose, a Vitamin A, a Vitamin D, a Vitamin B1 (thiamine), a Vitamin B12, a folate, a calcium, a Vitamin E, a Vitamin K, a zinc, a copper, a Vitamin B6, a Vitamin C, a homocysteine, an iron, a hemoglobin, a hematocrit, an insulin, a melanin, a hormone, a testosterone, an estrogen, a cortisol, a thyroxine, a triiodothyronine, a human growth hormone, an insulin-like growth factor, a thyroid stimulating hormone (TSH), a carotenoid, a cytokine, an interleukin, a chloride, a cholesterol, a lipoprotein, a triglyceride, a c-peptide, a creatinine, a creatine, a creatine kinase, a urea, a ketone, a peptide, a protein, an albumin, a bilirubin, a myoglobin, an ESR, a CRP, an IL6, an immunoglobin, a resistin, a ferritin, a transferrin, an antigen, a troponin, a gamma-glutamyltransferase (GGT), a lactate dehydrogenase (LD), an alanine aminotransferase, an alkaline phosphatase, or an aspartate aminotransferase.

18. The system of claim 11, wherein the processing further comprises calculating a reconstruction error of the low-rank tensor using an alternating minimum approach.

19. The system of claim 18, wherein calculating the reconstruction error comprises using the equation ||L||* + λ||S||1 such that M = L + S.
